(19) United States
(12) Patent Application Publication
(10) Pub. No.: US 2018/0024083 A1
(43) Pub. Date: Jan. 25, 2018

(54) GEL FOR SENSOR AND SENSOR

(71) Applicant: Seiko Epson Corporation, Tokyo (JP)

(72) Inventors: Satomi YOSHIOKA, Shiojiri (JP); Hiroshi YAGI, Tatsuno (JP)

(73) Assignee: Seiko Epson Corporation, Tokyo (JP)

(21) Appl. No.: 15/548,000

(22) PCT Filed: Jan. 13, 2016
(86) PCT No.: PCT/JP2016/000142

§ 371 Date: Aug. 1, 2017

(30) Foreign Application Priority Data
Feb. 12, 2015 (JP) 2015-025557

(51) Int. Cl. G01N 27/12; A61B 5/1468; G01N 33/487

(52) U.S. Cl. CPC G01N 27/12; G01N 33/48707; A61B 5/1468

(57) ABSTRACT

A gel for a sensor characterized by including a stimulus-responsive gel which expands or contracts in response to a given stimulus, and an electrically conductive substance which is included in the stimulus-responsive gel.

### BACKGROUND

### Technical Field

The present invention relates to a gel for a sensor and a sensor.

### Background Art

At present, as a method for obtaining in vivo biological information, a biochemical test in which the composition of blood obtained by blood collection is examined is generally performed. This test is mostly performed in medical institutions. Above all, a blood glucose sensor has been widely used in diabetic patients, and also a simple lactic acid sensor is getting widely used in athletes.

However, both are test methods involving blood collection using an invasive technique.

On the other hand, as a method using a non-invasive technique, a sensor targeting a component of sweat has been studied (see, for example, Wearable Technology for Bio-Chemical Analysis of Body Fluids During Exercise 30th Annual International IEEE EMBS Conference Vancouver, British Columbia, Canada, Aug. 20-24, 2008 and Novel lactate and pH biosensor for skin and sweat analysis based on single walled carbon nanotubes/Sensors and Actuators B 117 (2006) 308-313).

However, an enzyme used in such a method is generally expensive and is susceptible to temperature, humidity, etc., and therefore has problems that stable properties are hard to exhibit and the reliability of quantitativeness is low. In addition, the enzyme greatly varies in quality among production lots or manufacturers, and also has a problem that its properties change greatly over time.

An object of the invention is to provide a sensor capable of easily and stably performing detection of the intensity of a stimulus (the concentration or the like of a given component) in a wide region, and also to provide a gel for a sensor which can be favorably used for a sensor capable of easily and stably performing detection of the intensity of a stimulus (the concentration or the like of a given component) in a wide region.

### SUMMARY

A gel for a sensor of the invention is characterized by including

a stimulus-responsive gel which expands or contracts in response to a given stimulus, and

electrically conductive particles which are included in the stimulus-responsive gel.

According to this configuration, a gel for a sensor which can be favorably used for a sensor capable of easily and stably performing detection of the intensity of a stimulus (the concentration or the like of a given component) in a wide region can be provided.

In the gel for a sensor of the invention, it is preferred that as the electrically conductive substance, electrically conductive particles which are dispersed in the stimulus-responsive gel are included.

In the gel for a sensor of the invention, it is preferred that the electrically conductive particles are particles subjected to a surface treatment for improving the dispersibility in the stimulus-responsive gel.

In the gel for a sensor of the invention, it is preferred that the average particle diameter of the electrically conductive particles is 10 nm or more and 1000 μm or less.

In the gel for a sensor of the invention, it is preferred that the content of the electrically conductive particles with respect to 100 parts by volume of the stimulus-responsive gel when the stimulus-responsive gel is in an expanded state is 0.1 parts by volume or more and 65 parts by volume or less.

In the gel for a sensor of the invention, it is preferred that the gel for a sensor is provided with a recessed portion in a region coming into contact with an electrode.

In the gel for a sensor of the invention, it is preferred that in the recessed portion, the electrically conductive particles and the electrode are in contact with each other.

In the gel for a sensor of the invention, it is preferred that the electrical resistivity of the electrically conductive substance is 1.0×10⁻⁴ Ω·m or less.

In the gel for a sensor of the invention, it is preferred that the electrically conductive substance is constituted by a material containing one member or two or more members selected from the group consisting of a metal material, an electrically conductive metal oxide, a carbon material, and an electrically conductive polymer material.

A sensor of the invention is characterized by including

a stimulus-responsive gel,

an electrically conductive substance which is included in the stimulus-responsive gel, and

an electrode.

According to this configuration, a sensor capable of easily and stably performing detection of the intensity of a stimulus (the concentration or the like of a given component) in a wide region can be provided.

A sensor of the invention is characterized by including

the gel for a sensor of the invention, and

an electrode.

According to this configuration, a sensor capable of easily and stably performing detection of the intensity of a stimulus (the concentration or the like of a given component) in a wide region can be provided.

### BRIEF DESCRIPTION OF DRAWINGS

FIG. 1 is a schematic longitudinal cross-sectional view for illustrating a sensor of a first embodiment.

FIG. 2 is a schematic longitudinal cross-sectional view for illustrating a sensor of a second embodiment.

### DETAILED DESCRIPTION

Hereinafter, preferred embodiments will be described in detail with reference to the accompanying drawings.

### Sensor and Gel for Sensor

Hereinafter, a sensor (gel sensor) and a gel for a sensor will be described.

### First Embodiment

First, a sensor and a gel for a sensor of a first embodiment will be described.

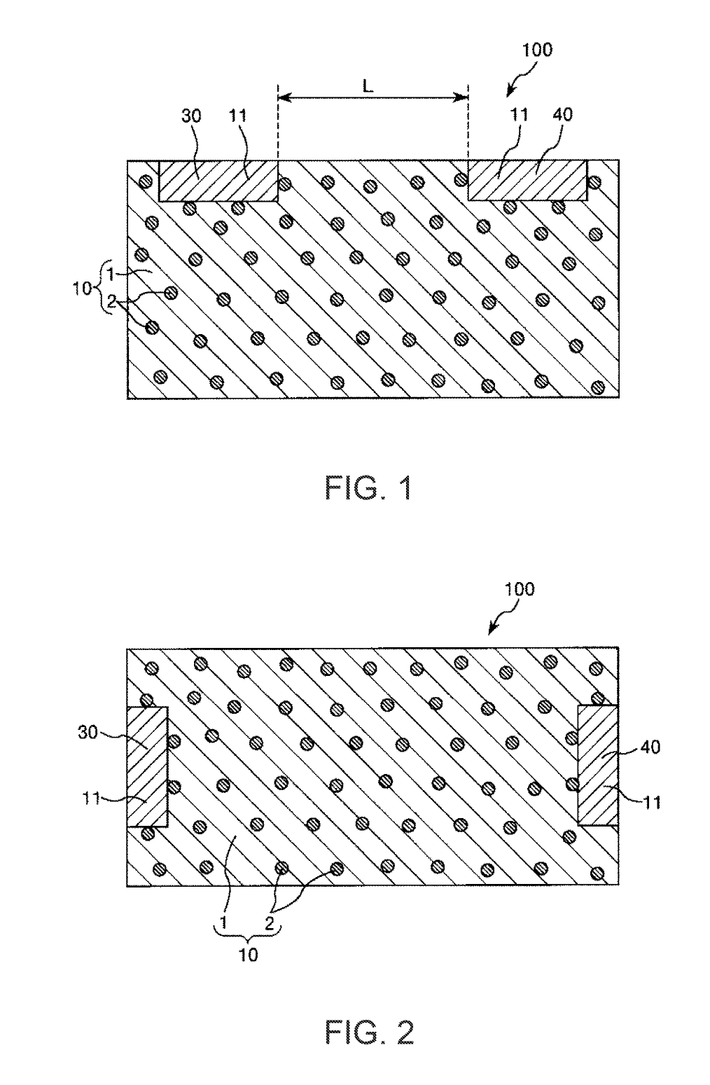

FIG. 1 is a schematic longitudinal cross-sectional view for illustrating a sensor of a first embodiment. Incidentally, in the following description, a case where the upper side in FIG. 1 is the observer side (viewpoint side) and the lower side in FIG. 1 is a side where a specimen is supplied will be mainly described.

As shown in FIG. 1, a sensor (gel sensor) 100 includes a gel for a sensor 10, a first electrode (electrode) 30, and a second electrode (electrode) 40. Then, the gel for a sensor 10 includes a stimulus-responsive gel 1 which expands or contracts in response to a given stimulus, and an electrically conductive substance 2 which is included in the stimulus-responsive gel 1.

According to such a configuration, a distance of the electrically conductive substances 2 which are included in the stimulus-responsive gel 1 (in the gel for a sensor 10) can be made different according to the expanded state or the contracted state of the stimulus-responsive gel 1, and as a result, a resistance value between the first electrode 30 and the second electrode 40 can be made different. That is, when the stimulus-responsive gel 1 (gel for a sensor 10) is in an expanded state (or in a state where the degree of expansion is large), the distance between the electrically conductive substances 2 is larger than when the stimulus-responsive gel 1 is in a contracted state (or in a state where the degree of expansion is small), and therefore, the resistance value of the gel for a sensor 10 connected to the electrodes (the first electrode 30 and the second electrode 40) is increased. Accordingly, by measuring the resistance value between the first electrode 30 and the second electrode 40, whether the stimulus-responsive gel 1 is in an expanded state or in a contracted state, that is, the intensity of a stimulus (the concentration or the like of a given component) can be easily and stably detected. In particular, the sensor (gel sensor) 100 is capable of easily and stably performing detection of the intensity of a stimulus in a wide region.

### Stimulus-Responsive Gel

The stimulus-responsive gel 1 expands or contracts in response to a given stimulus.

In this manner, since the stimulus-responsive gel 1 is a material that responds to a given stimulus, the resistance value of the gel for a sensor 10 connected to the electrodes (the first electrode 30 and the second electrode 40) can be changed according to the response (expansion or contraction) of the stimulus-responsive gel 1. As a result, by measuring the resistance value, the presence or absence of the stimulus, the intensity (amount or concentration) thereof, etc. can be detected.

The given stimulus to which the stimulus-responsive gel 1 responds varies depending on the constituent material or the like of the stimulus-responsive gel 1, however, examples thereof include various types of substances such as proteins, sugars, uric acid, lactic acid, various types of hormones, various types of ionic substances, and various types of metals, heat, and light.

In the configuration shown in the drawing, the stimulus-responsive gel 1 and the gel for a sensor 10 are in the form of a sheet.

According to this, while reducing the amount of the stimulus-responsive gel 1 and the amount of the electrically conductive substance 2 to a relatively small amount, the stimulus-responsive gel 1 and the gel for a sensor 10 can be efficiently brought into contact with a specimen, and also the detection of a given stimulus can be easily performed.

The volume of the stimulus-responsive gel 1 included in the sensor 100 is preferably 0.1 mm³ or more and 3600 mm³ or less, more preferably 0.2 mm³ or more and 900 mm³ or less.

According to this, while reducing the size of the sensor 100, the stimulus detection accuracy can be made more excellent. Further, also from the viewpoint of resource saving, the above configuration is preferred.

The electrical resistivity of the stimulus-responsive gel 1 alone is preferably 1.0×10⁻² Ω·m or more, more preferably 1.0×10⁻¹ Ω·m or more.

According to this, the effect of including the electrically conductive substance 2 is more remarkably exhibited, and the detection accuracy for the intensity of a stimulus (the concentration or the like of a given component) can be made more excellent in a wide region.

The electrical resistivity of the stimulus-responsive gel 1 alone preferably satisfies the conditions as described above when the stimulus-responsive gel 1 is either in an expanded state or in a contracted state, but more preferably satisfies the conditions as described above when the stimulus-responsive gel 1 is in both expanded state and contracted state.

According to this, the above-mentioned effect is more remarkably exhibited.

### Electrically Conductive Substance

In the stimulus-responsive gel 1, the electrically conductive substance 2 is included.

According to this, a distance between the electrically conductive substances 2 changes according to the response (expansion or contraction) of the stimulus-responsive gel 1 to a given stimulus, and the resistance value of the gel for a sensor 10 connected to the electrodes (the first electrode 30 and the second electrode 40) can be changed. As a result, by measuring the resistance value, the presence or absence of the stimulus, the intensity (amount or concentration) thereof, etc. can be detected.

The electrically conductive substance 2 may be any as long as it is constituted by a material having electrical conductivity, however, examples of the constituent material of the electrically conductive substance 2 include metal materials (for example, Au, Ag, Pt, Cu, an alloy containing at least one member selected from these, etc.), electrically conductive metal oxides (for example, ITO, etc.), carbon materials (for example, carbon black, etc.), and electrically conductive polymer materials (for example, PEDOT/PSS, a polythiophene-based material, a polyacetylene-based material, etc.).

By including the electrically conductive substance 2 constituted by such a material, while making the durability of the sensor 100 excellent, the percentage of change in the resistance value accompanying the expansion or contraction of the stimulus-responsive gel 1 can be further increased, and thus, the stimulus detection accuracy can be made more excellent. In addition, these materials are relatively inexpensive and can be stably obtained, and therefore are advantageous also from the viewpoint of suppression of the production cost of the sensor 100 and stable supply thereof.

The electrical resistivity of the electrically conductive substance 2 is preferably 1.0×10⁻⁴ Ω·m or less, more preferably 1.0×10⁴⁵ Ω·m or less, further more preferably 5.0×10⁻⁶ Ω·m or less.

According to this, the percentage of change in the resistance value accompanying the expansion or contraction of the stimulus-responsive gel 1 can be further increased, and thus, the stimulus detection accuracy can be made more excellent.

In this embodiment, as the electrically conductive substance 2, a plurality of electrically conductive particles which are dispersed in the stimulus-responsive gel 1 are included.

According to this, the stimulus detection accuracy can be easily and more stably made excellent. Further, the selection of the constituent material of the electrically conductive substance 2 can be widened.

The electrically conductive particle 2 may have a uniform composition in all regions, or may have a region having a different composition.

For example, the electrically conductive particle 2 may be a particle which has a core portion constituted by a first material and a coating film composed of at least one layer provided on the outer surface side of the core portion, or the like.

Further, the electrically conductive substance (electrically conductive particle) 2 may be a substance subjected to a surface treatment for improving the dispersibility in the stimulus-responsive gel 1.

According to this, an undesirable variation in the composition among the respective regions of the gel for a sensor 10 connected to the electrodes (the first electrode 30 and the second electrode 40) can be suppressed, and thus, the stability of the stimulus detection accuracy can be made more excellent.

Examples of a surface treatment agent which can be used in the surface treatment include agents having a hydroxy group, a carboxyl group, a sulfonic acid group, or a salt thereof, or the like in a partial structure.

As described above, in the case where the electrically conductive particle 2 has a region having a different composition, the electrically conductive particle 2 may have electrical conductivity to such an extent that the effect as described above can be exhibited as a whole, and a portion of the electrically conductive particle 2 may be constituted by an insulating material. For example, in the case where at least a portion of the base particle of the electrically conductive particle 2 is constituted by a material having electrical conductivity, a portion formed by the surface treatment (a surface-treated portion) may be constituted by an insulating material.

The average particle diameter of the electrically conductive particles 2 is not particularly limited, but is preferably 10 nm or more and 1000 μm or less, more preferably 20 nm or more and 500 μm or less.

According to this, in the stimulus-responsive gel 1 (in the gel for a sensor 10), the electrically conductive particles 2 can be more uniformly dispersed, and the stimulus detection accuracy can be made more excellent. Further, in the case where the average particle diameter of the electrically conductive particles 2 is relatively smaller (for example, 10 nm or more and 1000 nm or less), when a specific stimulus is received (for example, when a specific component is incorporated), a structural color caused by colloidal crystals or a change in the structural color is easily visually recognized, and therefore, not only can the stimulus be detected by measuring the resistance value as described above, but also the stimulus can be detected optically (visually). Further, since a structural color caused by colloidal crystals or a change in the structural color is easily visually recognized, for example, by the color tone thereof, also the quantitative determination of a specific stimulus (for example, a specific component) can be performed more easily and also more accurately.

Incidentally, in this description, the “average particle diameter” refers to an average particle diameter on a volume basis, and can be obtained by, for example, performing measurement using a particle size distribution analyzer employing a Coulter counter method (model: TA-II, manufactured by Coulter Electronics, Inc.) with an aperture of 50 μm for a dispersion liquid obtained by adding a sample to methanol and dispersing the sample therein for 3 minutes using an ultrasonic disperser.

The sensor 100 may include a plurality of types of electrically conductive substances 2.

The content of the electrically conductive substance 2 with respect to 100 parts by volume of the stimulus-responsive gel 1 when the stimulus-responsive gel is in an expanded state is preferably 0.1 parts by volume or more and 50 parts by volume or less, more preferably 0.5 parts by volume or more and 40 parts by volume or less.

According to this, the amount of change in the electrical resistivity according to the state (the expanded state or the contracted state) of the stimulus-responsive gel 1 can be further increased, and thus, the stimulus detection accuracy and detection sensitivity can be made more excellent.

In addition, the content of the electrically conductive particles 2 with respect to 100 parts by volume of the stimulus-responsive gel 1 when the stimulus-responsive gel is in a contracted state is preferably 30 parts by volume or more and 98 parts by volume or less, more preferably 45 parts by volume or more and 90 parts by volume or less.

According to this, the amount of change in the electrical resistivity according to the state (the expanded state or the contracted state) of the stimulus-responsive gel 1 can be further increased, and thus, the stimulus detection accuracy and detection sensitivity can be made more excellent.

A difference between the electrical resistivity in an expanded state (the maximum electrical resistivity) and the electrical resistivity in a contracted state (the minimum electrical resistivity) of the gel for a sensor 10 is preferably 1.0×10⁻⁵ Ω·m or more, more preferably 1.0×10⁻² Ω·m or more.

According to this, the stimulus detection accuracy can be made more excellent.

The thickness of the gel for a sensor 10 when the stimulus-responsive gel 1 is in an expanded state is not particularly limited, but is preferably 5 μm or more and 5000 μm or less, more preferably 7 μm or more and 1000 μm or less.

According to this, while making the durability and reliability of the sensor 100 sufficiently excellent, the thickness and size of the sensor 100 can be reduced. In addition, the flexibility of the sensor 100 can be made more excellent, and for example, even in the case where the sensor 100 is used in close contact with a living body or the like, the adhesiveness of the sensor 100 can be favorably maintained, and the stimulus detection accuracy can be stably made excellent.

### Electrode

To the gel for a sensor 10 including the stimulus-responsive gel 1 and the electrically conductive particles 2, the first electrode (electrode) 30 and the second electrode (electrode) 40 are connected.

According to this, the resistance value in the gel for a sensor 10 can be favorably measured, and from this measurement result, the presence or absence of a given stimulus received by the stimulus-responsive gel 1 or the intensity thereof can be determined.

The electrodes 30 and 40 are constituted by a material having electrical conductivity.

Examples of the constituent material of the electrodes 30 and 40 include metal materials (for example, Au, Ag, Pt, Cu, an alloy containing at least one member selected from these, etc.), electrically conductive metal oxides (for example, ITO, etc.), carbon materials (for example, carbon black, etc.), and electrically conductive polymer materials (for example, PEDOT/PSS, a polythiophene-based material, a polyacetylene-based material, etc.).

Above all, an electrically conductive metal oxide such as ITO has transparency and also has excellent durability, and therefore, the state of the gel for a sensor 10 can be favorably observed.

The thickness of the electrodes 30 and 40 is not particularly limited, but is preferably 5 μm or more and 5000 μm or less, more preferably 7 μm or more and 1000 μm or less.

According to this, while making the durability and reliability of the sensor 100 sufficiently excellent, the thickness and size of the sensor 100 can be reduced. In addition, the flexibility of the sensor 100 can be made more excellent, and for example, even in the case where the sensor 100 is used in close contact with a living body or the like, the adhesiveness of the sensor 100 can be favorably maintained, and the stimulus detection accuracy can be stably made excellent.

In this embodiment, a recessed portion 11 is provided in regions coming into contact with the electrodes 30 and 40 of the gel for a sensor 10.

According to this, the positioning of the gel for a sensor 10 and the electrodes 30 and 40 is facilitated, and thus, the stability of detection of a stimulus and the reliability of the sensor 100 can be made more excellent.

In addition, in the configuration shown in the drawing, the electrodes 30 and 40 are provided in contact with the electrically conductive particles 2 exposed on the surface of the gel for a sensor 10.

According to this, the stability of detection of a stimulus and the reliability of the sensor 100 can be made more excellent.

Further, in this embodiment, the electrodes 30 and 40 are provided on the surface of the gel for a sensor 10 on the side opposite to the side where the specimen is supplied.

According to this, for example, even in the case where the specimen is a liquid having electrical conductivity such as sweat, the occurrence of a short circuit due to the specimen existing outside the gel for a sensor 10 can be more effectively prevented, and thus, the reliability of the detection of a stimulus can be made more excellent.

A distance between the electrode 30 and the electrode 40 denoted by L in the drawing (inter-electrode distance) is preferably 0.1 mm or more and 50 mm or less, more preferably 0.2 mm or more and 30 mm or less.

According to this, while suppressing the increase in the size of the sensor 100, the reliability of the detection of a stimulus can be made more excellent.

In addition, the sensor 100 may include an absorbing member (not shown) which absorbs a specimen.

According to this, for example, in the case where an excess amount of a specimen exists outside the gel for a sensor 10, the excess amount of the specimen can be absorbed by the absorbing member, and even in the case where the specimen is a liquid having electrical conductivity such as sweat, the occurrence of a short circuit can be more effectively prevented, and thus, the reliability of the detection of the stimulus can be made more excellent. Further, in the case where a specimen is sequentially supplied (in the case where it is supplied continuously or intermittently), the previously supplied specimen is discharged, and the newly supplied specimen can be supplied to the stimulus-responsive gel 1, and therefore, a change in the amount of the stimulus over time can be found. That is, the detection of the accurate amount of the stimulus can be prevented from being disturbed due to mixing of the previously supplied specimen with the newly supplied specimen. In addition, for example, in the case where the supply of a liquid specimen is stopped or in the case where the supply amount of a specimen is significantly decreased, when the use environment of the sensor 100 is a low humidity environment or the like, the stimulus-responsive gel 1 can be prevented from being dried. As a result, even in an environment in which the stimulus-responsive gel 1 is easily dried, the detection of a given stimulus can be stably performed with high reliability over a relatively long period of time.

The absorbing member may be disposed in any place, but is preferably disposed in a place different from the surface of the gel for a sensor 10 on the side where the specimen is supplied.

According to this, while more favorably preventing the inhibition of supply of the specimen to the stimulus-responsive gel 1, the effect as described above can be exhibited.

In particular, the absorbing member is preferably disposed on the surface of the gel for a sensor 10 on the side opposite to the side where the specimen is supplied.

According to this, the effect as described above is more remarkably exhibited, and in particular, in the case where the specimen is sequentially supplied (in the case where it is supplied continuously or intermittently), the previously supplied specimen is discharged, and the newly supplied specimen can be favorably supplied to the stimulus-responsive gel 1, and therefore, a change in the amount of the stimulus over time can be favorably found.

In addition, the absorbing member is preferably disposed so as not to come into contact with a plurality of electrodes.

According to this, while exhibiting the effect as described above, for example, even in the case where the specimen is a liquid having electrical conductivity such as sweat, the occurrence of a short circuit due to the specimen existing outside the gel for a sensor 10 can be more effectively prevented, and thus, the reliability of the detection of the stimulus can be made more excellent.

Examples of the absorbing member include members constituted by a woven fabric, a non-woven fabric, a cloth-like material such as felt, a porous material, or the like.

Examples of a preferred constituent component of the absorbing member include cellulose materials and water-absorbing polymers, however, a cellulose material is particularly preferred. Such a material has moderate hydrophilicity, and therefore, for example, in the case where the specimen contains water (for example, a body fluid such as sweat), the specimen can be favorably absorbed in the material, and also water contained in the absorbed specimen can be favorably evaporated from the material.

### Second Embodiment

Next, a sensor of a second embodiment will be described.

FIG. 2 is a schematic longitudinal cross-sectional view for illustrating a sensor of a second embodiment. In the following description, different points from the above-mentioned embodiment will be mainly described, and the description of the same matter will be omitted.

The sensor (gel sensor) 100 of this embodiment has the same configuration as that of the above-mentioned embodiment except that the place where the electrodes 30 and 40 are provided is different. That is, in the above-mentioned embodiment, the electrodes 30 and 40 are provided on the surface of the gel for a sensor 10 on the side opposite to the side where the specimen is supplied, however, in the sensor (gel sensor) 100 of this embodiment, the electrodes 30 and 40 are provided in a side surface portion of the gel for a sensor 10. In this manner, the place where the electrodes are provided may be any place. In particular, as in this embodiment, by providing the electrodes 30 and 40 in a side surface portion of the gel for a sensor 10 in the form of a sheet including the stimulus-responsive gel 1 and the electrically conductive particles 2, the thickness of the sensor 100 can be reduced.

In the configuration shown in FIG. 2, the electrodes 30 and 40 are provided only in a portion in the thickness direction of the gel for a sensor 10, but may be provided throughout the thickness direction of the gel for a sensor 10.

### Constituent Material of Stimulus-Responsive Gel

Next, the constituent material of the stimulus-responsive gel constituting the sensor will be described.

The stimulus-responsive gel may be constituted by any material as long as it responds to a given stimulus, but is generally constituted by a material containing a polymer material having a crosslinked structure and a solvent.

### Polymer Material

The polymer material constituting the stimulus-responsive gel is an important component for detecting a specific stimulus, and the structure thereof varies depending on the type of the stimulus to be detected.

The polymer material constituting the stimulus-responsive gel is not particularly limited, and can be selected according to the stimulus to be detected.

Hereinafter, specific examples of the polymer material constituting the stimulus-responsive gel will be described.

As the polymer material constituting the stimulus-responsive gel, for example, a polymer material obtained by reacting a monomer, a polymerization initiator, a crosslinking agent, etc. can be used.

Examples of the monomer include acrylamide, N-methylacrylamide, N-isopropylacrylamide, N,N-dimethylacrylamide, N,N-dimethylaminopropylacrylamide, various quaternary salts of N,N-dimethylaminopropylacrylamide, acryloylmorpholine, various quaternary salts of N,N-dimethylaminoethylacrylate, acrylic acid, various alkyl acrylates, methacrylic acid, various alkyl methacrylates, 2-hydroxyethylmethacrylate, glycerol monomethacrylate, N-vinylpyrrolidone, acrylonitrile, styrene, polyethylene glycol diacrylate, 1,6-hexanediol diacrylate, neopentyl glycol diacrylate, tripropylene glycol diacrylate, polypropylene glycol diacrylate, 2,2-bis[4-(acryloxydiethoxy)phenyl]propane, 2,2-bis[4-(acryloxypolyethoxy)phenyl]propane, 2-hydroxy-1-acryloxy-3-methacryloxypropane, 2,2-bis[4-(acryloxypolypropoxy)phenyl]propane, ethylene glycol dimethacrylate, diethylene glycol dimethacrylate, triethylene glycol dimethacrylate, polyethylene glycol dimethacrylate, 1,3-butylene glycol dimethacrylate, 1,6-hexanediol dimethacrylate, neopentyl glycol dimethacrylate, polypropylene glycol dimethacrylate, 2-hydroxy-1,3-dimethacryloxypropane, 2,2-bis[4-(methacryloxyethoxy)phenyl]propane, 2,2-bis[4-(methacryloxyethoxydiethoxy)phenyl]propane, 2,2-bis[4-(methacryloxyethoxypolyethoxy)phenyl]propane, trimethylolpropane trimethacrylate, tetramethylolmethane trimethacrylate, trimethylolpropane triacrylate, tetramethylolmethane triacrylate, tetramethylolmethane tetraacrylate, dipentaerythritol hexaacrylate, N,N′-methylenebisacrylamide, N,N′-methylenebismethacrylamide, diethylene glycol diallyl ether, and divinylbenzene.

Further, examples of a functional group which can interact with a sugar include a boronic acid group (particularly, a phenylboronic acid group), and therefore, a monomer having a boronic acid group may be used. Examples of such a boronic acid group-containing monomer include acryloylaminobenzeneboronic acid, methacryloylaminobenzeneboronic acid, and 4-vinylbenzeneboronic acid.

Further, in the case where an ionic substance (particularly, an ionic substance containing a calcium ion) is detected as a stimulus (specific component), a crown ether group-containing monomer (particularly, a benzocrown ether group-containing monomer) such as 4-acrylamidobenzo-18-crown 6-ether, acryloyl aminobenzocrown ether, methacryloyl aminobenzocrown ether, or 4-vinylbenzocrown ether can be preferably used as the monomer.

Further, in the case where an ionic substance such as sodium chloride is detected as a stimulus (specific component), 3-acrylamidophenylboronic acid, vinylphenylboronic acid, acryloyloxyphenylboronic acid, N-isopropylacrylamide (NIPAAm), ethylenebisacrylamide, N-hydroxyethylacrylamide, or the like can be preferably used as the monomer. In particular, in the case where anionic substance such as sodium chloride is detected as a stimulus (specific component), it is preferred to use one monomer or two or more monomers selected from the group consisting of 3-acrylamidophenylboronic acid, vinylphenylboronic acid, and acryloyloxyphenylboronic acid, and one monomer or two or more monomers selected from the group consisting of N-isopropylacrylamide (NIPAAm), ethylenebisacrylamide, and N-hydroxyethylacrylamide in combination as the monomer.

Further, in the case where lactic acid is detected as a stimulus (specific component), 3-acrylamidophenylboronic acid, vinylphenylboronic acid, acryloyloxyphenylboronic acid, N-isopropylacrylamide (NIPAAm), ethylenebisacrylamide, N-hydroxyethylacrylamide, or the like can be preferably used as the monomer. In particular, in the case where lactic acid is detected as a stimulus (specific component), it is preferred to use one monomer or two or more monomers selected from the group consisting of 3-acrylamidophenylboronic acid, vinylphenylboronic acid, and acryloyloxyphenylboronic acid, and one monomer or two or more monomers selected from the group consisting of N-isopropylacrylamide (NIPAAm), ethylenebisacrylamide, and N-hydroxyethylacrylamide in combination as the monomer.

The polymerization initiator can be appropriately selected according to, for example, the polymerization method thereof. Specific examples thereof include compounds which generate radicals by ultraviolet light including hydrogen peroxide, persulfates such as potassium persulfate, sodium persulfate, and ammonium persulfate, azo-based initiators such as 2,2′-azobis(2-amidinopropane) dihydrochloride, 2,2′-azobis(N,N′-dimethyleneisobutylamidine) dihydrochloride, 2,2′-azobis{2-methyl-N-[1,1-bis(hydroxymethyl)-2-hydroxyethyl]propionamide}, 2,2′-azobis[2-(2-imidazolin-2-yl)propane] dihydrochloride, 4,4′-azobis(4-cyanovaleric acid), 2,2′-azobisisobutyronitrile, 2,2′-azobis(2,4′-dimethylvaleronitrile), benzophenone, 2,2-dimethoxy-1,2-diphenylethan-1-one, 1-hydroxycyclohexyl phenyl ketone, 2-hydroxy-2-methyl-1-phenylpropan-1-one, 2,4,6-trimethylbenzoyl diphenylphosphine oxide, 1-[4-(2-hydroxyethoxy)-phenyl]-2-hydroxy-2-methyl-1-propan-1-one, and the like, and compounds which generate radicals by light with a wavelength of 360 nm or more such as substances obtained by mixing a thiopyrylium salt-based, merocyanine-based, quinoline-based, or styrylquinoline-based dye with 2,4-diethyl thioxanthone, isopropyl thioxanthone, 1-chloro-4-propoxythioxanthone, 2-(3-dimethylamino-2-hydroxypropoxy)-3,4-dimethyl-9H-thioxanthon-9-one methochloride, 2-methyl-1[4-(methylthio)phenyl]-2-morpholinopropane-1,2-benzyl-2-dimethylamino-1-(4-morpholinophenyl)-butanone-1, bis(cyclopentadienyl)-bis(2,6-difluoro-3-(pyl-1-yl) titanium, or a peroxy ester such as 1,3-di(t-butylperoxycarbonyl)benzene or 3,3′,4,4′-tetra-(t-butylperoxycarbonyl)benzophenone. Further, hydrogen peroxide or a persulfate can also be used as a redox-based initiator in combination with, for example, a reducing substance such as a sulfite or L-ascorbic acid, an amine salt, or the like.

As the crosslinking agent, a compound having two or more polymerizable functional groups can be used, and specific examples thereof include ethylene glycol, propylene glycol, trimethylolpropane, glycerin, polyoxyethylene glycol, polyoxypropylene glycol, polyglycerin, N,N′-methylenebisacrylamide, N,N-methylene-bis-N-vinylacetamide, N,N-butylene-bis-N-vinylacetamide, tolylene diisocyanate, hexamethylene diisocyanate, allylated starch, allylated cellulose, diallyl phthalate, tetraallyloxyethane, pentaerythritol triallyl ether, trimethylolpropane triallyl ether, diethylene glycol diallyl ether, and triallyl trimellitate.

The stimulus-responsive gel may include a plurality of different types of polymer materials.

The content of the polymer material in the stimulus-responsive gel is preferably 0.7 mass % or more and 36.0 mass % or less, more preferably 2.4 mass % or more and 27.0 mass % or less.

### Solvent

By including the solvent in the stimulus-responsive gel, the above-mentioned polymer material can be favorably gelled.

As the solvent, any of various types of organic solvents and inorganic solvents can be used. Specific examples thereof include water; various types of alcohols such as methanol and ethanol; ketones such as acetone; ethers such as tetrahydrofuran and diethyl ether; amides such as dimethylformamide; chain aliphatic hydrocarbons such as n-pentane, n-hexane, n-heptane, and n-octane; alicyclic hydrocarbons such as cyclohexane and methylcyclohexane; and aromatics such as benzene, toluene, and xylene, however, in particular, a solvent containing water is preferred.

The stimulus-responsive gel may include a plurality of different types of components as the solvent.

The content of the solvent in the stimulus-responsive gel is preferably 30 mass % or more and 95 mass % or less, more preferably 50 mass % or more and 90 mass % or less.

### Another Component

The stimulus-responsive gel may include a component other than the above-mentioned components (another component).

For example, insulating particles having a given size may be included. According to this, when a specific stimulus is received (for example, when a specific component is incorporated), a structural color caused by colloidal crystals or a change in the structural color is easily visually recognized, and therefore, not only can the stimulus be detected by measuring the resistance value as described above, but also the stimulus can be detected optically (visually). Further, since a structural color caused by colloidal crystals or a change in the structural color is easily visually recognized, for example, by the color tone thereof, also the quantitative determination of a specific stimulus (for example, a specific component) can be performed more easily and also more accurately.

Examples of a constituent material of the insulating particles include inorganic materials such as silica and titanium oxide; and organic materials (polymers) such as polystyrene, polyester, polyimide, polyolefin, poly(methyl (meth)acrylate), polyethylene, polypropylene, polyether sulfone, nylon, polyurethane, polyvinyl chloride, and polyvinylidene chloride, however, the insulating particles are preferably silica fine particles. According to this, the stability of the shape of the insulating particles and the like can be made particularly excellent, and the durability, reliability, and the like of the stimulus-responsive gel can be made particularly excellent. Further, silica fine particles are relatively easily available as particles having a sharp particle size distribution (monodispersed fine particles), and therefore are advantageous also from the viewpoint of stable production and supply of the stimulus-responsive gel.

The shape of the insulating particle is not particularly limited, but is preferably a spherical shape. According to this, a structural color caused by colloidal crystals or a change in the structural color is visually recognized, and therefore, the detection of a specific stimulus can be more easily performed.

The average particle diameter of the insulating particles is not particularly limited, but is preferably 10 nm or more and 1000 nm or less, more preferably 20 nm or more and 500 nm or less.

According to this, a structural color caused by colloidal crystals or a change in the structural color is more easily visually recognized, and therefore, an optical (visual) detection of a specific stimulus can be more easily and also more reliably performed. In addition, since a structural color caused by colloidal crystals or a change in the structural color is more easily visually recognized, for example, by the degree of the change in the color tone, also the quantitative determination of a specific stimulus can be more easily and also more accurately performed.

The stimulus-responsive gel may include a plurality of different types of insulating particles.

The content of the insulating particles in the stimulus-responsive gel is preferably 1.6 mass % or more and 36 mass % or less, more preferably 4.0 mass % or more and 24 mass % or less.

### Use of Sensor

The sensor can easily detect a given stimulus (for example, a specific component), and therefore can be used as, for example, a sensor for determining whether or not a specific substance is contained in a test subject (a specimen) or measuring the concentration of a specific substance contained in a test subject.

Further, the sensor can easily detect the amount of a specific component incorporated in the stimulus-responsive gel, and therefore can also be favorably used as a separation and extraction unit that separates and extracts a specific substance contained in a test subject. That is, at a stage where the amount of a specific component incorporated in the stimulus-responsive gel is saturated or almost saturated, the contact thereof with the test subject is stopped, and according to need, it can be replaced by another sensor. By doing this, the specific component can be collected from the test subject without any waste.

More specific use of the sensor include, for example, sensors for biological substances (for example, various types of cells such as cancer cells and blood cells, proteins such as antibodies (including glycoproteins and the like), etc.), sensors for components (for example, lactic acid, uric acid, sugars, etc.) contained in body fluids or substances secreted outside the body (for example, blood, saliva, sweat, urine, etc.), separation and extraction units for biological substances (particularly, trace biological substances such as hormones, etc.), separation and extraction units for metals (particularly, rare metals, noble metals, etc.), sensors for antigens (allergic substances) such as pollens, separation and extraction units for poisons, toxic substances, environmental pollutants, etc., sensors for viruses, bacteria, etc., sensors for components contained in soils, sensors for components contained in waste fluids (including drain water), sensors for components contained in foods, sensors for components contained in water (for example, salts and the like contained in brackish waters, rivers, paddies, etc.), cell culture monitors, and the like.

Further, the sensor is preferably a sensor to be used in close contact with the skin of a living body.

The skin of a living body generally has a complicated rugged shape, however, as described above, the sensor has excellent shape followability, and therefore can be favorably brought into close contact with the skin of a living body. Further, in the case where the sensor is used in close contact with the skin of a living body (for example, in the case where a component contained in sweat during an exercise is detected as a stimulus (specific component), or the like), it is presumed that a large external force such as vibration or impact is applied to the sensor. However, even in such a case that a relatively large external force is applied to the sensor, a specific component (given stimulus) can be accurately detected. Therefore, the effect is more remarkably exhibited in the case where the sensor is used in close contact with the skin of a living body.

Further, the sensor is also favorably applicable to reduction in size and weight. Accordingly, the sensor is suitable for use in the method as described above.

### Method of Using Sensor

Hereinafter, one example of a method of using the sensor will be specifically described.

First, the sensor 100 when the stimulus-responsive gel 1 is in a state where it does not receive a given stimulus (for example, in the case where the given stimulus is a specific component, a state where the stimulus-responsive gel 1 does not incorporate the specific component) is prepared.

Subsequently, the electrodes 30 and 40 are electrically connected to a unit that measures an electrical resistance, and the resistance value in a state where the stimulus-responsive gel 1 does not receive a given stimulus is measured (initialization). At this time, in the case where the given stimulus is a specific substance, for example, calibration may be performed using a standard solution containing the specific substance at a given concentration. By doing this, the stimulus detection accuracy can be further enhanced.

Thereafter, the detection of the stimulus is performed in a state where the specimen and the sensor 100 can come into contact with each other.

By performing the measurement in such a manner, the detection of the intensity of a stimulus (the concentration or the like of a given component) can be easily and stably performed in a wide region.

The sensor 100 is preferably a sensor which constitutes a part of a wearable device or a sensor which is used by being connected to a wearable device.

According to this, for example, the burden on a user accompanying the detection of a stimulus can be reduced, and the sensor 100 can be favorably used, for example, even during an exercise or the like. In addition, a user or the like can favorably confirm the detection result displayed on a display section. Further, such a configuration is preferred also from the viewpoint of fashionability or the like.

Examples of the wearable device include a watch-type device.

In addition, for example, after the detection of a stimulus, the gel for a sensor 10 may be washed as needed. By doing this, the gel for a sensor 10 can be favorably reused, and the lifetime of the gel for a sensor 10 and the sensor 100 can be prolonged. The washing of the gel for a sensor 10 can be performed using, for example, a liquid which does not contain a specific component (given stimulus).

Further, when the sensor 100 is not used, the gel for a sensor 10 may be stored in a state where the gel for a sensor 10 is in contact with a liquid which does not contain a specific component (given stimulus). By doing this, the gel for a sensor 10 can be favorably stored, and the lifetime of the gel for a sensor 10 and the sensor 100 can be prolonged.

Hereinabove, preferred embodiments have been described, however, the invention is not limited thereto.

For example, the sensor may have a configuration other than those described above.

For example, the sensor may include an adhesive layer for adhering the sensor to a given position.

Further, in the above-mentioned embodiments, the electrode has been described as an electrode which is in contact with the gel for a sensor, however, at least one intermediate layer may be provided between the electrode and the gel for a sensor. According to this, for example, the adhesiveness between the gel for a sensor and the electrode can be made more excellent.

Further, the sensor may have a partition wall that divides the gel for a sensor into a plurality of cells.

Further, the sensor may include a housing member that houses the gel for a sensor. According to this, for example, the gel for a sensor can be favorably protected from an external force or the like, and thus, the detection of a stimulus can be more stably performed.

Further, in the above-mentioned embodiments, a case where the stimulus-responsive gel is in the form of a sheet and also the sensor as a whole is in the form of a sheet has been representatively described, however, the stimulus-responsive gel may be in a form other than a sheet, and also the shape of the sensor may be any form, for example, a block form, a string form, a cylindrical form, or the like.

Further, in the above-mentioned embodiments, a case where the electrically conductive substance is a particle (electrically conductive particle) in the form of a spherical shape has been mainly described, however, the shape of the electrically conductive substance may be any. In addition, the electrically conductive substance may be included in a state of being dissolved in the stimulus-responsive gel.

In addition, in the sensor, some constituent members may be configured to be replaceable or may be configured to be detachable and reattachable. For example, the absorbing member may be configured to be replaceable, or detachable and reattachable. According to this, even if a large amount of a solid component (for example, a specific component or the like) is adhered to the absorbing member, the sensor can be used by replacing the absorbing member, or detaching and washing the absorbing member, or the like. In this manner, the lifetime of the sensor can be prolonged.

The entire disclosure of Japanese Patent Application No. 2015-025557, filed Feb. 12, 2015 is expressly incorporated by reference herein.

1. A gel for a sensor, characterized by comprising:
a stimulus-responsive gel which expands or contracts in response to a given stimulus; and
an electrically conductive substance which is included in the stimulus-responsive gel.

2. The gel for a sensor according to claim 1, wherein as the electrically conductive substance, electrically conductive particles which are dispersed in the stimulus-responsive gel are included.

3. The gel for a sensor according to claim 2, wherein the electrically conductive particles are particles subjected to a surface treatment for improving the dispersibility in the stimulus-responsive gel.

4. The gel for a sensor according to claim 2, wherein the average particle diameter of the electrically conductive particles is 10 nm or more and 1000 μm or less.

5. The gel for a sensor according to claim 2, wherein the content of the electrically conductive particles with respect to 100 parts by volume of the stimulus-responsive gel when the stimulus-responsive gel is in an expanded state is 0.1 parts by volume or more and 65 parts by volume or less.

6. The gel for a sensor according to claim 1, wherein the gel for a sensor is provided with a recessed portion in a region coming into contact with an electrode.

7. The gel for a sensor according to claim 6, wherein in the recessed portion, the electrically conductive substance and the electrode are in contact with each other.

8. The gel for a sensor according to claim 1, wherein the electrical resistivity of the electrically conductive substance is 1.0×10⁻⁴ Ω·m or less.

9. The gel for a sensor according to claim 1, wherein the electrically conductive substance is constituted by a material containing one member or two or more members selected from the group consisting of a metal material, an electrically conductive metal oxide, a carbon material, and an electrically conductive polymer material.

10. A sensor, characterized by comprising:
a stimulus-responsive gel;
an electrically conductive substance which is included in the stimulus-responsive gel; and
an electrode.

11. A sensor, characterized by comprising:
the gel for a sensor according to claim 1; and
an electrode.
